(19) United States
(12) Patent Application Publication
(10) Pub. No.: US 2002/0098111 A1
(43) Pub. Date: Jul. 25, 2002

(54) Vaporizer

(76) Inventors: Nick N. Nguyen, Costa Mesa, CA (US); James P. Kohler, Laguna Hills, CA (US)

Correspondence Address: Philip S. Johnson, Esq., Johnson & Johnson, One Johnson & Johnson Plaza, New Brunswick, NJ, 08933-7003, US

(21) Appl. No.: 09/728,973

(22) Filed: Dec. 4, 2000

(51) Int. Cl. A61L 9/02

(52) U.S. Cl. 422/28

(57) ABSTRACT

A vaporizer provides enhanced removal of non-vaporizable components from a liquid to be vaporized. Flow of vaporizing fluid through the vaporizer follows a circuitous path to allow the non-vaporizable components to adhere to surfaces in the vaporizer rather than leave in the vaporized flow.

### FIELD OF THE INVENTION

The present invention relates to vaporizers and methods of vaporizing, and more particularly to vaporizers for chemical vapor sterilization systems and for a method of vaporizing with sterilants.

### BACKGROUND

Vapor based chemical sterilization systems are a popular alternative to steam sterilization. They typically allow sterilization at lower temperatures than is possible with steam, thereby allowing sterilization of articles sensitive to high temperatures. Several such systems are commercially available, such as the STERRAD Brand hydrogen peroxide gas/plasma sterilization system.

In this system, a sterilization chamber is brought to low pressures, approximately one Torr, and liquid hydrogen peroxide is admitted into the chamber and vaporized into the low pressure. The hydrogen peroxide vapor diffuses to articles placed within the chamber. After a time, an electromagnetic field or other means is employed to ignite a plasma of the hydrogen peroxide vapor and after the plasma inducing field is removed, the constituents reassemble to form oxygen and water. Such systems are more fully described in U.S. Pat. Nos. 4,643,876 and 4,756,882 which are incorporated herein by reference.

Solutions of hydrogen peroxide and other liquid sterilants typically contain non-vaporizable constituents; for instance, in a typical 59% concentration hydrogen peroxide solution, trace quantities of chemicals such as transition metal salts and organic free radical scavengers, are present to stabilize the liquid solution. Upon vaporization of the hydrogen peroxide solution, these chemicals are left as solid particulates. If no effort is made to separate and collect these constituents, they may become deposited upon items in the sterilization chamber. Most of these constituents are harmless, and their presence is merely unsightly and/or perhaps provides a false impression that these sterilization processes were not complete. However, in some sterilization processes, these constitutes may either be harmful to the instruments and to the patient. Accordingly, it is desirable to remove such constituents prior to releasing the vaporized hydrogen peroxide or other sterilant to the sterilization chamber

U.S. Pat. No. 6,106,772, which is incorporated herein by reference, by Kohler and Williams, addresses this problem by providing an impingement plate outside of the vaporizer in the chamber upon which the stream of hydrogen peroxide which is being vaporized impinges prior to the contacting the devices or load to be sterilized in the sterilization chamber. In this fashion, a portion of the non-vaporizable constituents adheres to the plate rather than depositing onto the load in the sterilization chamber. While such system provides a marked improvement over no control of non-vaporizable constituents, small amount of such non-vaporizable constituents may still deposit on the load in the sterilization chamber. Accordingly, it would be desirable to provide a system and method for collecting such constituents with a higher degree of efficiency.

The STERRAD 200 brand hydrogen peroxide/gas plasma type sterilizer employs a vaporizer in which the vaporizing hydrogen peroxide follows a path formed by a series of annular fins in a cylindrical chamber creating a series of torus-like spaces, and wherein each fin has an opening therethrough offset from the opening in the adjacent fins whereby to provide a series of direction changes through the vaporizer.

### SUMMARY OF THE INVENTION

The present applicants have discovered that by providing a flow restriction, residence time within the vaporizer is enhanced and the efficiency of the vaporizer is also enhanced.

A vaporizer according to the present invention vaporizes a sterilant from its liquid phase in a vapor phase sterilization system having a pressure below atmospheric pressure. The vaporizer comprises an inlet to receive the sterilant in its liquid phase, an outlet to discharge the sterilant in its vapor phase, a circuitous path between the inlet and the outlet to collect non-vaporizable ingredients of the sterilant, and a flow restriction.

Preferably, the circuitous path comprises a plurality of baffles. The circuitous path can comprise an inner tube positioned concentrically within an outer tube, the circuitous path including a first portion in a first direction between the inner tube and the outer tube and a second portion in a second opposite direction through the inner tube. The circuitous path comprises at least one portion in which an effective cross-sectional area of the portion increases by at least 89% to decrease the speed of the sterilant passing therethrough. The circuitous path preferably comprises at least two turns, each of which are at least 90 degrees.

The flow restriction can comprise an orifice having a cross-sectional area no greater than 44.1% of a cross-sectional area of the circuitous path immediately upstream of the orifice. Preferably, restriction can retain the vapor within the vaporizer for at least 17 milliseconds, and more preferably for at least 26 milliseconds.

A method of providing a vapor phase sterilant to a sterilization chamber, according to the present invention, comprising the steps of creating temperature and pressure conditions within a vaporizer sufficient to vaporize the sterilant and admitting the sterilant, in its liquid phase, into the vaporizer and vaporizing the sterilant. The sterilant passes through a circuitous path where non-vaporizable components of the sterilant collect on surfaces forming the circuitous path. The sterilant, in its vapor phase, passes through a flow restriction which increases residency within the circuitous path and enhances efficiency of collecting non-vaporizable components. The vaporized sterilant passes out of the vaporizer.

The non-vaporizable components can comprise stabilizing compounds for the liquid phase of the sterilant. The sterilant can comprise hydrogen peroxide.

Preferably, at least 50%, and more preferably at least 75%, of the non-vaporizable components are removed from the sterilant prior to the step of passing the sterilant out of the vaporizer.

### BRIEF DESCRIPTION OF THE EDRAWINGS

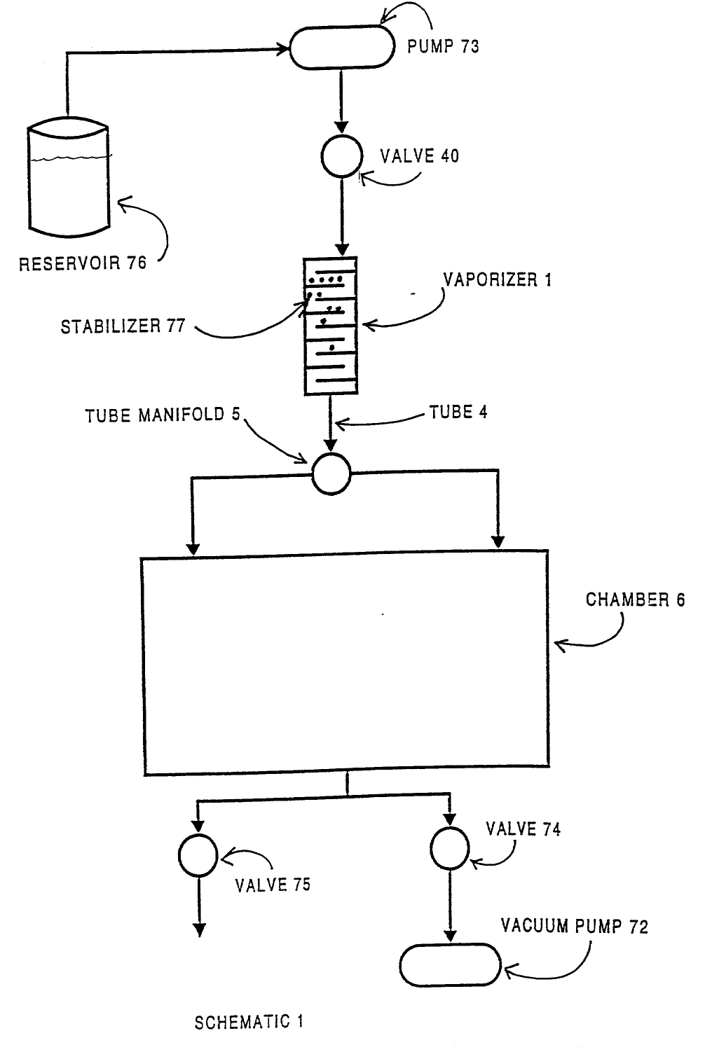

FIG. 1 is a flow diagram of a sterilization system employing a vaporizer according to the present invention;

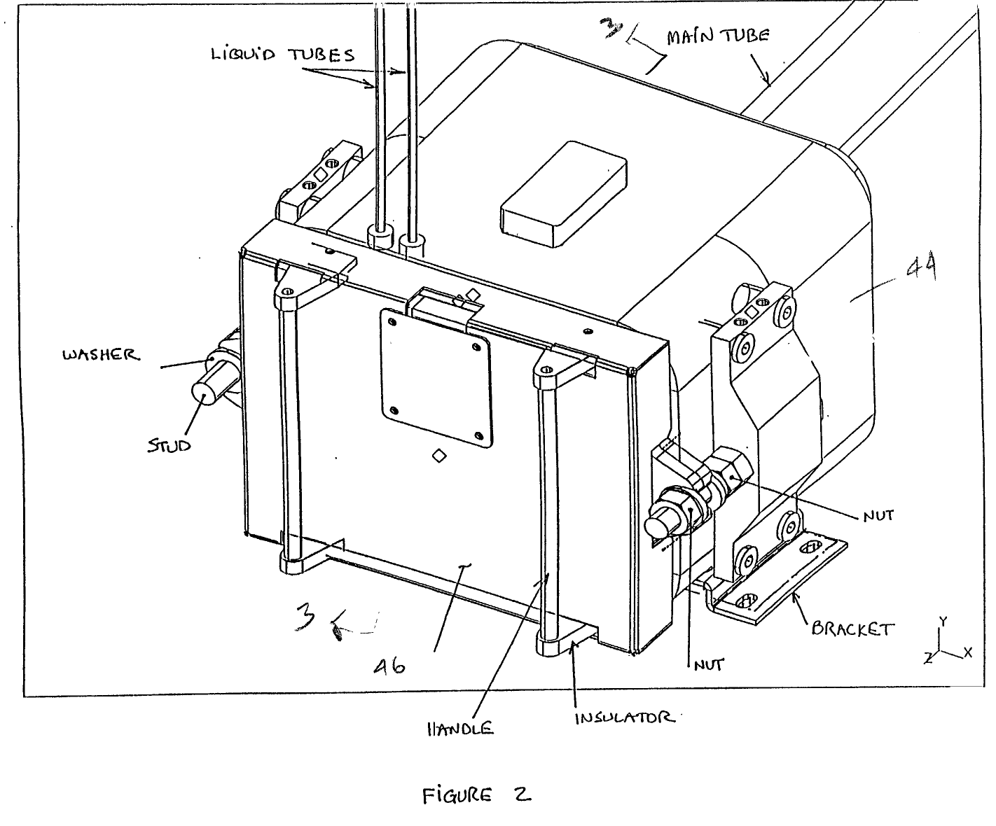

FIG. 2 is a perspective view of a first embodiment of the vaporizer of FIG. 1;

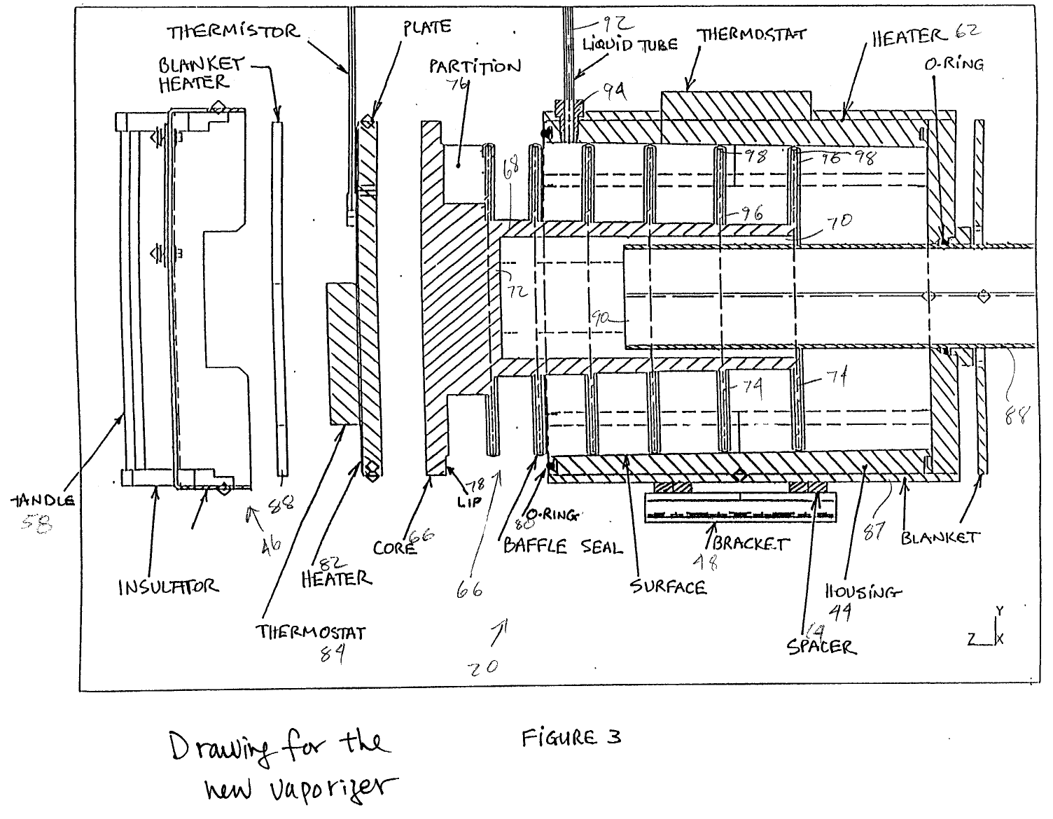

FIG. 3 is an exploded sectional view taken along lines 3--3 of FIG. 2, in which the core is partially removed;

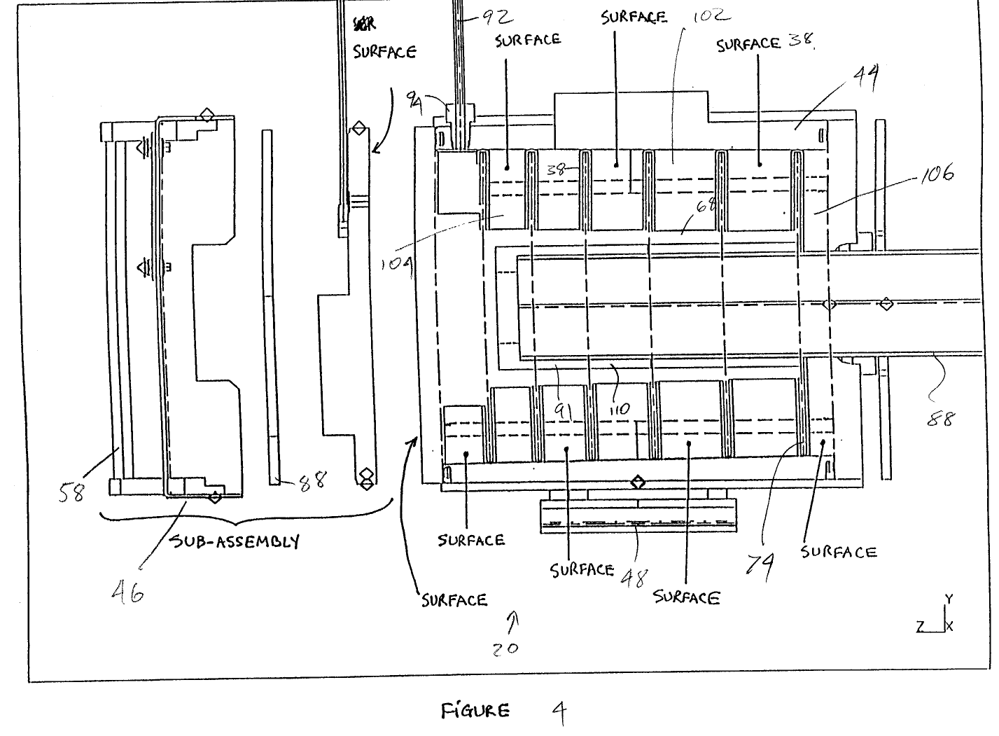

FIG. 4 is an exploded sectional view taken along lines 3--3 of FIG. 2, in which the core is not removed;

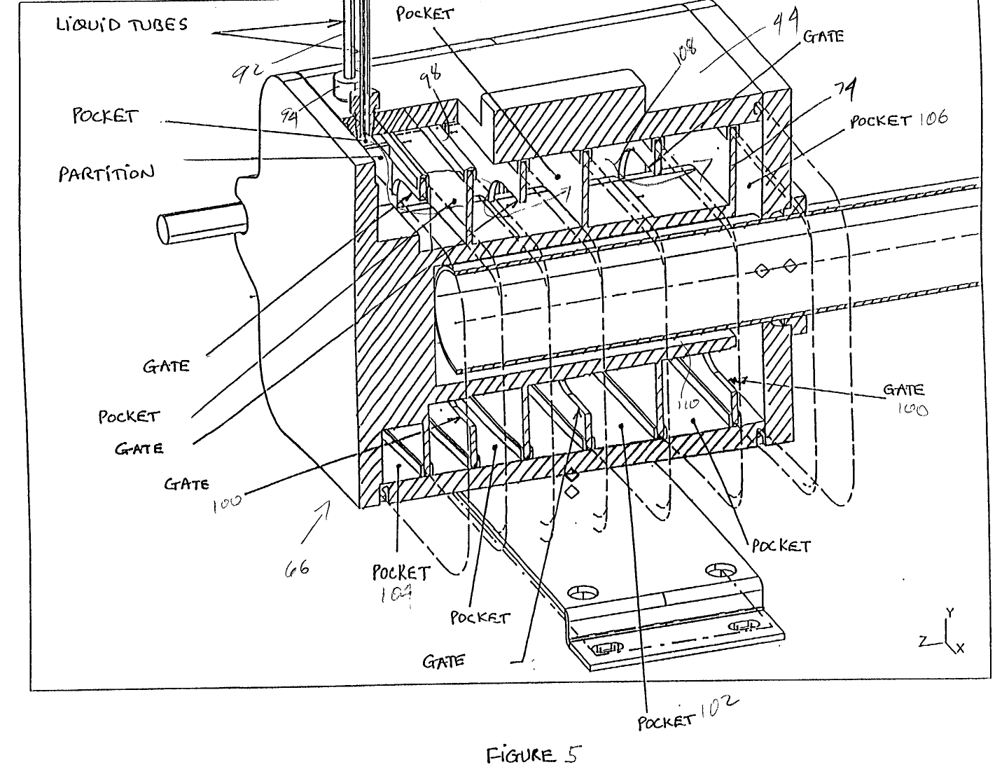

FIG. 5 is a perspective sectional view taken along lines 3--3 of FIG. 2;

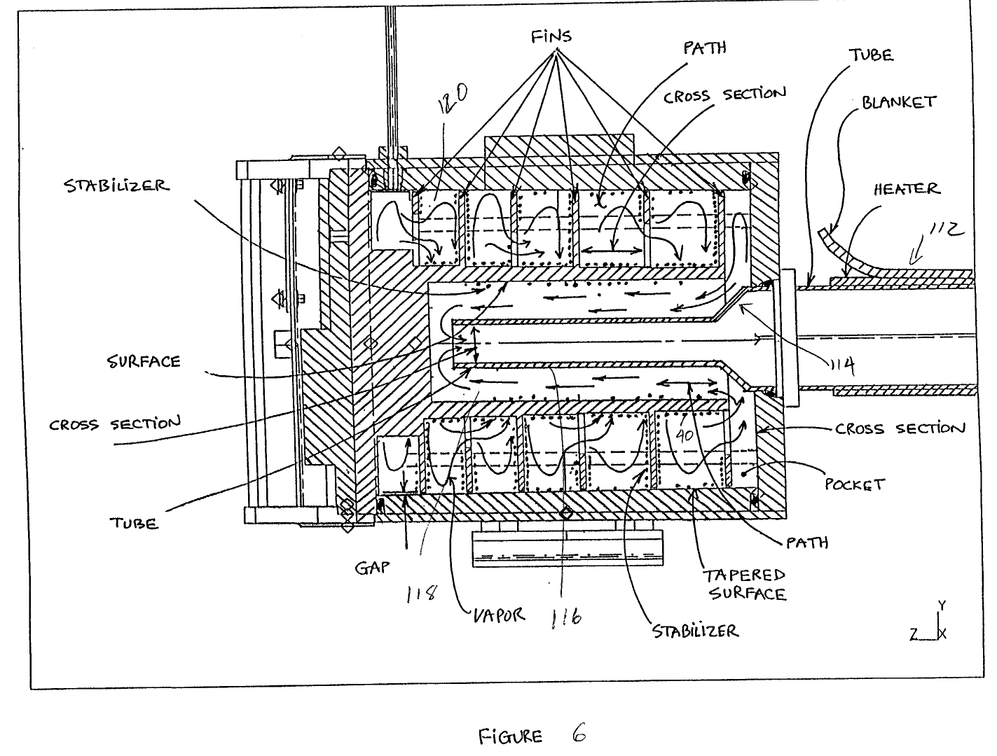

FIG. 6 is a sectional view of a second embodiment of a vaporizer according to the present invention;

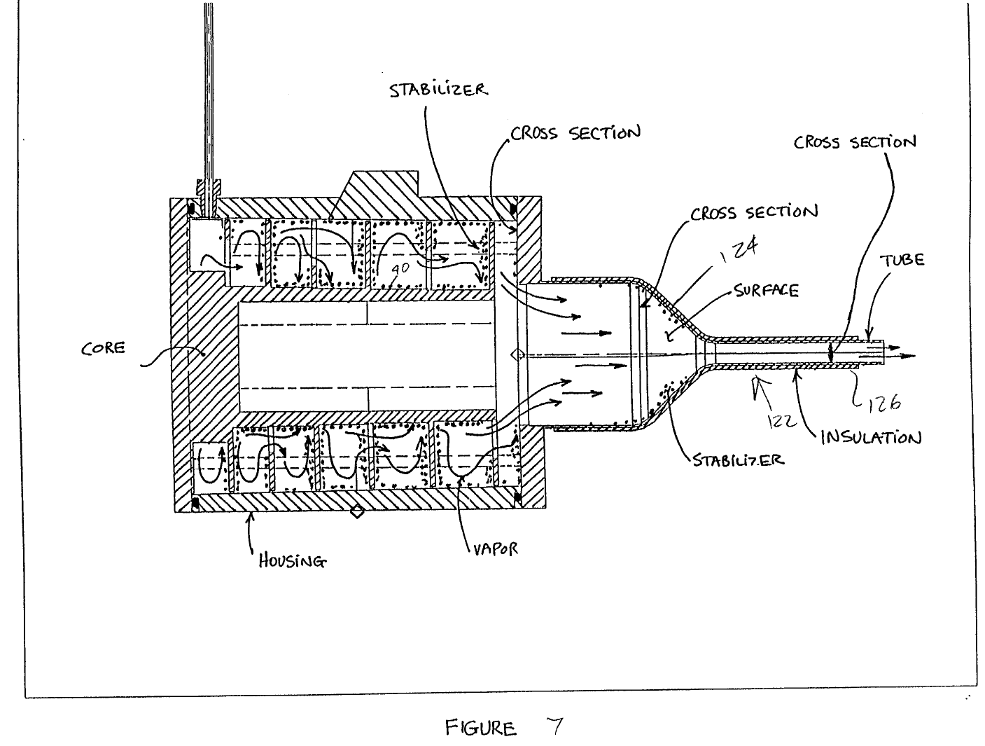

FIG. 7 is a sectional view of a third embodiment of a vaporizer according to the present invention;

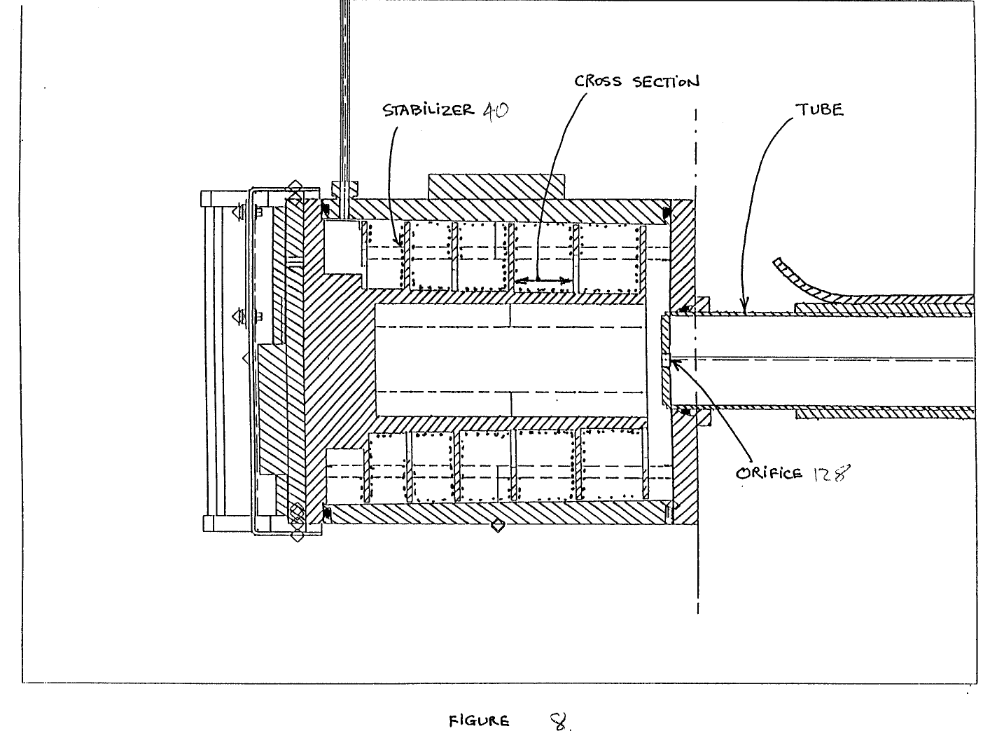

FIG. 8 is a sectional view of a fourth embodiment of a vaporizer according to the present invention;

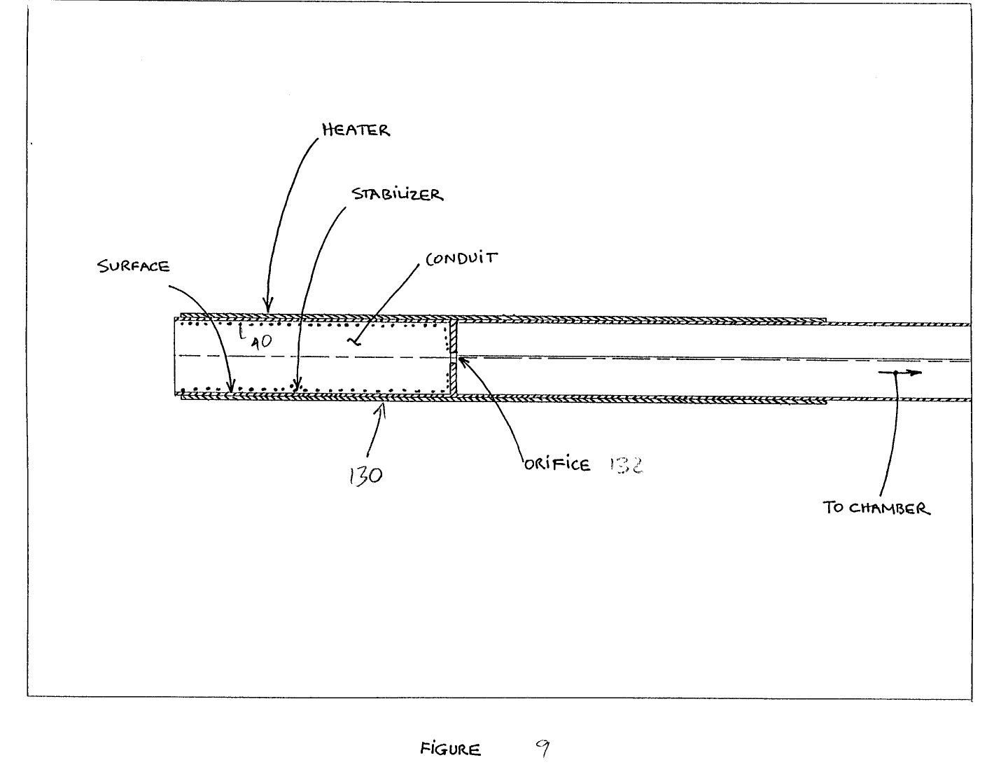

FIG. 9 is a sectional view of an outlet tube of a fifth embodiment of a vaporizer according to the present invention;

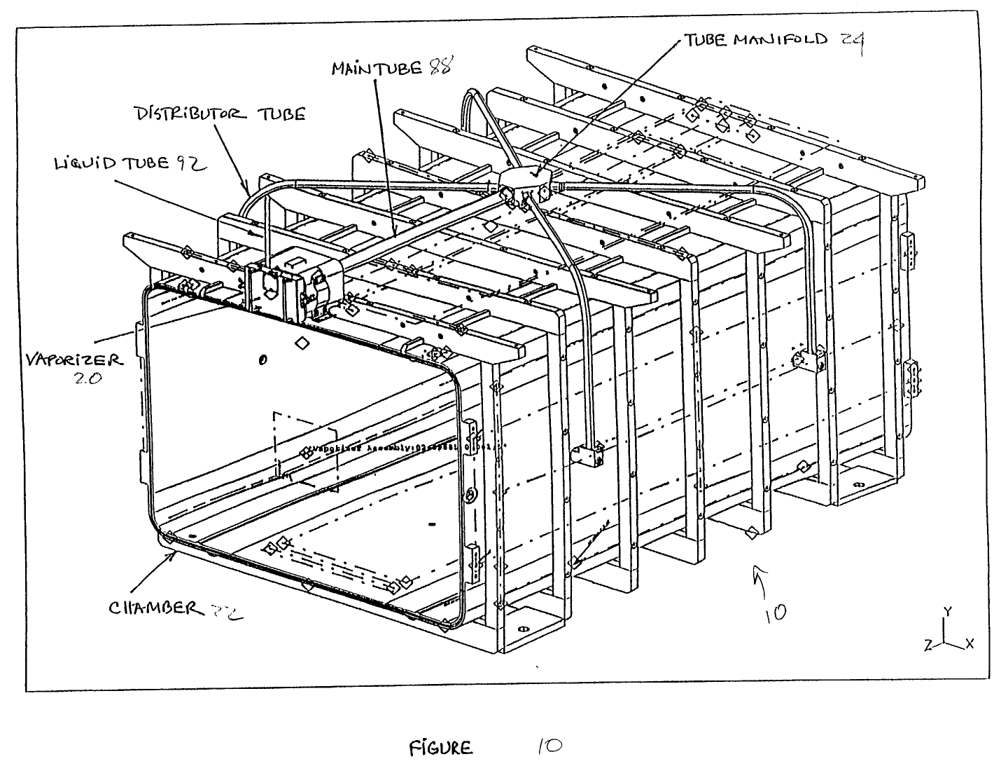

FIG. 10 is a perspective view of the system of FIG. 1; and

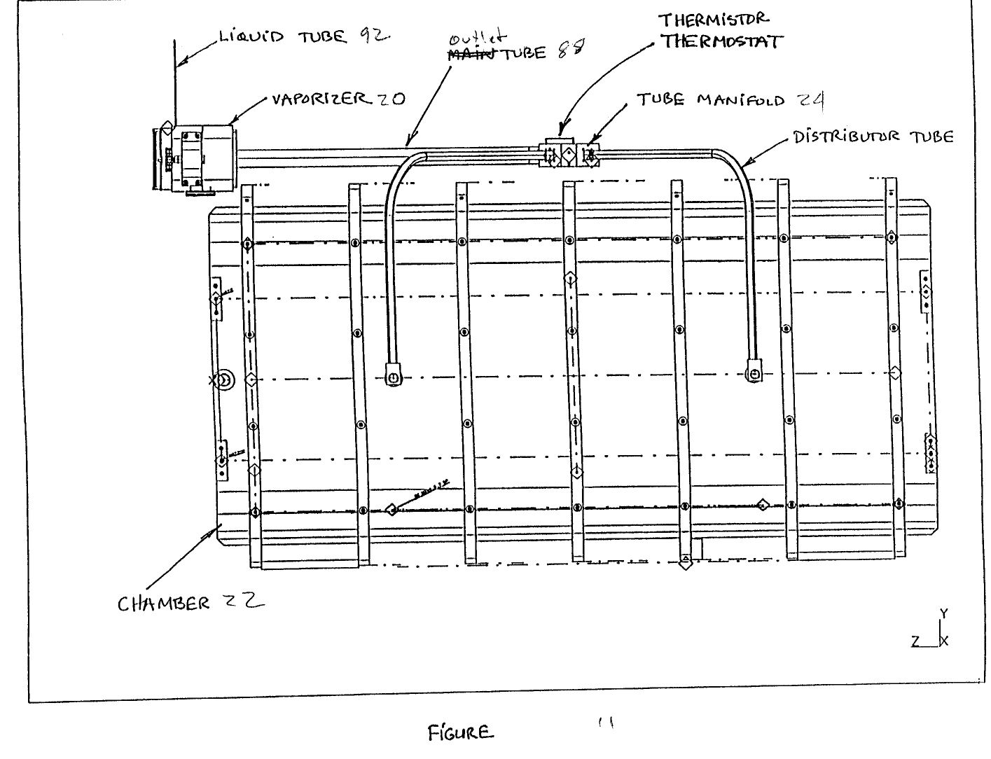

FIG. 11 is a side elevation view of the system of FIG. 10.

### DESCRIPTION

FIG. 1 illustrates in schematic format of a vapor phase sterilization system 10 and components for providing sterilant thereto. Liquid sterilant, such as a 59% solution of hydrogen peroxide and water, is stored within a reservoir 14. A pump 16 and valve 18 control flow of sterilant 12 from the reservoir 14 to a vaporizer 20. The vaporizer 20 connects to a sterilization chamber 22 through a manifold 24. A vacuum pump 26 and a valve 28 provide means for drawing a vacuum on the chamber 22 and a vent valve 30 allow venting of the chamber 22 to atmosphere.

Before admission of the sterilant 12, a vacuum is drawn on the chamber 22 by the vacuum pump 26. Typically, the vacuum is approximately 1 Torr. The vaporizer 20 is fluidly connected to the chamber 22 and is, therefore, effectively at the same pressure initially as the chamber 22 with the exception of the flow induced pressure drops therebetween. Liquid sterilant 12 enters the vaporizer through an inlet 32 and immediately begins vaporizing due to the low pressure and heated vaporizer therein.

It travels a circuitous path 34 therethrough, such as created by a series of baffles 36 or other flow direction changing objects which provide a plurality of directional changes, thereby allowing the flow of vaporizing sterilant 12 to impinge upon surfaces 38 with the vaporizer 20 as it passes therethrough. Such impingement causes non-vaporizable components 40 in the sterilant 12 to deposit upon these impingement surfaces 38. As a sterilant 12 exits the vaporizer 20 through its exit 42, a fairly large proportion of the non-vaporizable components 40 are left adhered to the impingement surfaces 38 within the vaporizer. Thus, as the sterilant 12 travels through the manifold 24 into the chamber 22 it is relatively free of non-vaporizable constituents.

FIG. 2 shows one embodiment of the vaporizer 20 according to the present invention. It comprises a housing 44 having a removable panel 46. The housing 44 fits into a mounting bracket 48. Threaded fittings 50 on the bracket 48 connect to lugs 52 and 54 on the housing 44 and panel 46 respectively and are held by means of nuts 56. Handles 58 are provided on the panel 46 for removing the panel.

Turning also to FIGS. 3 and 4, it can be seen that the entire housing 44 is insulated by a blanket 60, which may comprise any suitable insulation. An electric heater 62 lies between the blanket 60 and the housing 44. The spacers 64 between the housing 44 and the mounting bracket 48 also help to reduce heat loss from the housing 44.

A core 66 fits within the housing 44. The core 66 comprises a cylinder 68 having an open end 70 and closed end 72 with a plurality of annular fins 74 extending radially therefrom. The fins 74 extend toward the housing 44 but do not actually touch the housing. A partition 76 having an annular lip 78 attaches to the cylinder closed end 72 and seals against the housing 44 by means of an O-ring 80. A core heater 82 having a thermostat 84 and thermister 86 attached to the partition 76 to heat the core 66. An insulating blanket 87 covers the heater 82. All of this is enclosed by the removable panel 46 so that the core 66 can be easily removed for cleaning.

The core cylinder 68 fits over an outlet tube 88 which extends into the housing 44. The outlet tube 88 has an outside diameter slightly smaller than the inside diameter of the core cylinder 68 and has an open end 90 which sits adjacent to but does not abut the cylinder closed end 72. The tight fit between the outlet tube 88 and core cylinder 68 creates a flow restriction 91.

A pair of liquid tubes 92 enter the housing 44 adjacent the partition 76 and are preferably attached through a fitting 94.

Turning also now to FIG. 5, a gasket 96 covers distal edges 98 of each of the fins 74 to seal the fins 74 against the housing 44. A series of openings 100 through the fins adjacent the cylinder 68 are provided and are offset from each other on adjoining fins 74 so that the gases may flow past the fins 74 through the openings 100, but in doing so make frequent directional changes. The fins 74 create a series of spaces or pockets 102 with an inlet pocket 104 adjacent the liquid tubes 92 and a terminal pocket 106 adjacent the cylinder open end 70. Liquid entering the vaporizer 20 through the liquid tubes 92 is vaporized and flows along a circuitous path 108 through the openings 100 to the terminal pocket 106 and then into a space 110 between the cylinder 68 and outlet tube 88. It enters the space 110 through the cylinder open end 70. Flow then proceeds into the outlet tube 88 through its open end 90. Along the way, such flow impinges upon many surfaces leaving behind deposits of non-vaporizable components 40.

FIG. 6 illustrates an alternative embodiment of the invention. This embodiment employs an outlet tube 112 having a reducing section 114 at the interface with the housing 44 and which leads to a smaller diameter section 116 within the cylinder 68. Accordingly, a space 118 between the outlet tube 112 and cylinder 68 has a larger cross-sectional area than in the previous embodiment. This reduces the velocity of the flow in the space 118 and increases residence time so as to allow a higher portion of the non-vaporizable components 40 to come out of the sterilant 12. The narrow diameter of the small diameter section 116 also enhances this effect as the cross-sectional area in the small diameter section 116 is less than the cross-sectional area in the space 118 or in a space 120 between the cylinder 68 and housing 44 thereby acting as a flow restriction.

FIG. 7 illustrates a further embodiment in which an outlet tube 122 does not extend into the housing but has a high area ratio (greater than or equal to 3:1) reducing section 124 leading to a very narrow outlet portion 126 thereby providing a flow restriction. This large flow restriction substantially decreases the velocity in the remainder vaporizer 20 thereby allowing a longer residence time and a higher degree of separation of the non-vaporizable components 40 for a given size of the vaporizer 20. Alternatively, the size of the vaporizer 20 can be reduced while maintaining the same level of efficiency in removing non-vaporizable components 40.

FIG. 8 illustrates the same concept but employs an orifice 128 rather than a reducing section to provide a flow restriction.

FIG. 9 illustrates an outlet tube 130 having an orifice 132 in the middle thereof.

TABLE 1 illustrates how a flow restriction can enhance the efficiency of the vaporizer 20 in collecting the non-vaporizable components 40. It illustrates the performance difference of a vaporizer configured according to that shown in FIG. 6 having two different size small diameter sections 116. By reducing the diameter by 50%, the collection efficiency was increased from 76% to 100%.

TABLE 1
FLUID VELOCITY AND
RESIDENCE TIME IN THE VAPORIZER AT 70° C. WITH
TWO DIFFERENT SIZES OF OUTLET TUBES INJECTION
OF 15 ML OF 59 WT% HYDROGEN PEROXIDE SOLUTION
0.75 in OD Tube

| | Cross | Average | Stabilizer | Residence |
|---|---|---|---|---|
| Space | Sectional | Velocity | Collected, | Time, |
| Number | Area in² | ft/sec | g | milliseconds |
| 120 | 1.8 | 158 | 2.67 | 26 |
| 118 | 4.7 | 59 | 0.06 | |
| 116 | 0.4 | 747 | 0 | |

| | |
|---|---|
| Total Collected, g | 2.73 |
| Measured from Solution, g | 2.60 |
| % Recovered | 100 |

Ratio of (space 116/space 118) = 0.4/4.7 = 8.5%
Ratio of (space 118-space 120)/(space 120) = 161%

1.5 in OD Tube

| | Cross | Average | Stabilizer | Residence |
|---|---|---|---|---|
| Space | Sectional | Velocity | Collected, | Time, |
| Number | Area in² | ft/sec | g | milliseconds |
| 120 | 1.8 | 245 | 1.96 | 17 |
| 118 | 3.4 | 127 | 0.02 | |
| 116 | 1.5 | 286 | 0 | |

| | |
|---|---|
| Total Collected, g | 1.98 |
| Measured from Solution, g | 2.60 |
| % Recovered | 76 (75% in Space 120) |

Ratio of (space 116/space 118) = 1.5/3.4 = 44.1%
Ratio of (space 118-space 120)/(space 120) = 89%

The residence time of vapor retained in the vaporizer can be calculated according to the following equations:

t=(L/v ) ×1000,

v=(W ×144) / (ρ×A),

where

t=calculated residence time, milliseconds,

L=measured length of flow path, ft, p1 v=calculated vapor velocity, ft/sec,

W=measured mass flow rate, lb/sec,

ρ=calculated vapor density, (P ×MW)/(R ×T), lb/ft³,

P=measured upstream pressure in vaporizer, psia,

MW=calculated vapor molecular weight, g/mole,

R=gas constant, mmHg-l/mole ° K.,

T=measured vapor temperature, ° K.,

A=measured cross sectional area for flow in the vaporizer, in².

The 17 milliseconds residence time for the 1.5 inches OD tube can be calculated with the follow measured data.

L=4.1 ft

W=1.4×10⁻³ lb/sec

P=0.125 lb_{f}/in²

T=343° K.

A=1.75 in²

ρ=(P ×MW) / (R ×T)

=(0.125 lb_{f}/in²×760 mmHg/atm ×25 g/mole ×28.32 l/ft³) / (14.7 lb_{f}/in²−atm ×62.36 mmHg-l/mole ° K. ×343° K. ×454 g/lb)

=4.7×10⁻⁴ lb/ ft³

v=W/(ρA)

=(1.4×10⁻³ lb/sec ×144 in² /ft² ) / (4.7×10⁻⁴ lb/ ft³×1.75 in²)

=245 ft/sec

t=(L/v) ×1000

=4.1 ft ×1000 milliseconds/sec / 245 ft/sec =17 milliseconds

FIGS. 10 and 11 illustrate the system 10 with the vaporizer 20 located atop the sterilizer chamber 22 and showing the manifold 24 leading from the vaporizer 20 into various locations into the sterilization chamber.

After a number of cycles, a sufficient amount of non-vaporizable components 40 will become deposited on the components within the vaporizer 20 and it will be desirable to remove these deposits. Preferably, the housing 44 tapers slightly from where the panel 46 attaches to where the outlet tube 88 leaves so that when the panel 46 is removed the core 66 can be slid out of the housing 44 more easily. If it becomes stuck, the nuts 56 can be turned to drive the core 66 out of the housing 44.

The invention now being fully described, it will be apparent to one of ordinary skill in the art that many modifications and changes can be made thereto without departing from the spirit or scope of the invention as defined in the following claims.

What is claimed is:

1. A vaporizer for vaporizing a sterilant from its liquid phase in a vapor phase sterilization system having a pressure below atmospheric pressure, said vaporizer comprising:
an inlet whereby to receive the sterilant in its liquid phase;
an outlet whereby to discharge the sterilant in its vapor phase;
a circuitous path between the inlet and the outlet whereby to collect non-vaporizable ingredients of the sterilant; and
a flow restriction between the circuitous path and the outlet.

2. A vaporizer according to claim 1 wherein the circuitous path comprises a plurality of baffles.

3. A vaporizer according to claim 1 wherein the circuitous path comprises an inner tube positioned concentrically within an outer tube, the circuitous path including a first portion in a first direction between the inner tube and the outer tube and a second portion in a second opposite direction through the inner tube.

4. A vaporizer according to claim 1 wherein the circuitous path comprises at least one portion in which an effective cross-sectional area of the portion increases by at least 89% whereby to decrease the speed of the sterilant passing therethrough.

5. A vaporizer according to claim 1 wherein the flow restriction comprises an orifice having a cross-sectional area no greater than 44.1% of a cross-sectional area of the circuitous path immediately upstream of the orifice.

6. A vaporizer according to claim 1 wherein the circuitous path comprises at least two turns, each of which are at least 90 degrees.

7. A vaporizer according to claim 1 wherein the restriction can retain the vapor within the vaporizer for at least 17 milliseconds.

8. A vaporizer according to claim 7 wherein the restriction can retain the vapor within the vaporizer for at least 26 milliseconds.

9. A method of providing a vapor phase sterilant to a sterilization chamber comprising the steps of:
creating temperature and pressure conditions within a vaporizer sufficient to vaporize the sterilant;
admitting the sterilant, in its liquid phase, into the vaporizer and vaporizing the sterilant;
passing the sterilant through a circuitous path and collecting non-vaporizable components of the sterilant on surfaces forming the circuitous path;
passing the sterilant, in its vapor phase, through a flow restriction; and
passing the sterilant, in its vapor phase, out of the vaporizer.

10. A method according to claim 9 wherein the step of passing the sterilant through a circuitous path comprises passing the sterilant past a plurality of baffles.

11. A method according to claim 9 wherein the step of passing the sterilant through the circuitous path comprises passing the sterilant in a first direction through an inner tube positioned concentrically within an outer tube and in a second opposite direction between the inner tube and the outer tube.

12. A method according to claim 9 wherein the step of passing the sterilant through a circuitous path comprises passing the sterilant through at least one portion in which an effective cross-sectional area of the portion increases by at least 89% thereby decreasing the speed of the sterilant passing therethrough.

13. A method according to claim 9 wherein the step of passing the sterilant through the circuitous path comprises passing the sterilant through an orifice having a cross-sectional area no greater than 44.1% of a cross-sectional area of the circuitous path immediately upstream of the orifice.

14. A method according to claim 9 wherein the step of passing the sterilant through the circuitous path comprises having the sterilant make at least two turns, each of which are at least 90 degrees.

15. A method according to claim 9 wherein the non-vaporizable components comprise stabilizing compounds for the liquid phase of the sterilant.

16. A method according to claim 16 wherein the sterilant comprises hydrogen peroxide.

17. A method according to claim 9 wherein at least 75% of the non-vaporizable components are removed from the sterilant prior to the step of passing the sterilant out of the vaporizer.

18. A method according to claim 17 wherein substantially all of the non-vaporizable components are removed from the sterilant prior to the step of passing the sterilant out of the vaporizer.

19. A method according to claim 9 wherein the sterilant remains within the vaporizer for at least 17 milliseconds.

20. A method according to claim 19 wherein the sterilant remains within the vaporizer for at least 26 milliseconds.
